(19) United States
(12) Patent Application Publication
(10) Pub. No.: US 2024/0386994 A1
(43) Pub. Date: Nov. 21, 2024

(54) IDENTIFYING AND QUANTIFYING RELATIONSHIPS AMONGST CELLS AND GENES

(71) Applicant: Accenture Global Solutions Limited, Dublin (IE)

(72) Inventor: Maziyar Baran Pouyan, Peta;i,a, CA (US)

(21) Appl. No.: 18/659,766

(22) Filed: May 9, 2024

Related U.S. Application Data
(60) Provisional application No. 63/502,172, filed on May 15, 2023.

(51) Int. Cl. G16B 20/00; G06N 20/20; G16B 25/10; G16B 40/20; G16B 45/00

(52) U.S. Cl. CPC G16B 20/00; G06N 20/20; G16B 25/10; G16B 40/20; G16B 45/00

(57) ABSTRACT

The present disclosure relates to a system, computer readable medium, and method for identifying and quantifying relationships amongst cells and genes. Aspects of Random Forest are leveraged in a way that is unconventional. Traditionally, Random Forest, a machine learning algorithm, is used to combine the output of multiple decision trees to reach a single result. However, in the disclosed system and computer implemented method using the structure of decision trees created by Random Forest to determine relationships between genes and cells. The relationships may include similarity between pairs of cells and/or interactions of genes within individual cells.

### CROSS-REFERENCE TO RELATED APPLICATION

This application claims the benefit of U.S. Provisional Patent Application Ser. No. 63/502, 172 filed on May 15, 2023, and titled “Identifying and Quantifying Relationships Amongst Cells and Genes”, the disclosure of which is incorporated by reference herein in its entirety.

### TECHNICAL FIELD

The present disclosure generally relates to identifying relationships amongst cells and genes.

### BACKGROUND

Traditionally, ribonucleic acid (RNA) has been analyzed by bulk sequencing, which involves analyzing the genome of a cell population, such as a cell culture, a tissue, an organ, or entire organism, rather than individual cells. Bulk sequencing produces an average genome. Single cell sequencing produces genomes of individual cells that form a cell population. The advancement of single cell sequencing improves the ability to identify more granular properties of individual cells and to measure the RNA expression of a considerable amount of single cells simultaneously, resulting in noticeable progress in the knowledge of cellular structure. Current approaches only take into account the pair-wise similarity between gene expression profiles of two cells. In these methods, no dependencies or interactions between various genes are taken into account (e.g., Euclidean metric). However, gene-gene interactions (gene regulatory network) play a crucial impact in addition to gene expression levels in Identifying cell-cell similarities.

Typically, a cell's biological structure is a very complex and nonlinear dynamical system. Gene expressions in such a system are variables from a dynamical standpoint and can differ even inside the same cell if they are assessed at various times or under various circumstances. Contrarily, the measured patterns of gene expression are the consequence of transcriptional networks or gene connections, which are stable through time and under varying conditions. As a result, a cell's network can more accurately describe the biological system or condition of the cell.

Different cells have different developmental origins and stages, which can influence a gene regulatory network. Due at least in part to the complexity of cell development, no practical and accurate method exists for extracting complex non-linear gene interaction for a single cell.

There is a need in the art for a system and method that addresses the shortcomings discussed above.

### SUMMARY

A system and computer implemented method Identifying and quantifying relationships amongst cells and genes is disclosed. The disclosed system and computer implemented leverage aspects of Random Forest in a way that is unconventional. Traditionally, Random Forest, a machine learning algorithm, is used to combine the output of multiple decision trees to reach a single result. However, in the disclosed system and computer implemented method includes generating decision trees of a Random Forest, and using the structure of the decision trees for an entirely different purpose without regard for the single result. The disclosed system and computer implemented method leverages the structure of the decision trees to determine relationships between genes and cells. For example, the relationships may include the similarities between pairs of cells. In another example, relationships may include the interactions amongst genes of an individual cell.

In some embodiments, the disclosed system and computer implemented method includes generating bipartite graphs based on only a portion of each decision tree from a Random Forest. The bipartite graphs can be merged to form a consolidated bipartite graph that represents dependencies between cells and genes. The disclosed system and computer implemented method may include applying a node embedding algorithm to the consolidated bipartite graph to generate feature embeddings for each cell node. These embeddings can provide biologically meaningful information about cells.

These feature embeddings can be used to find the similarity between cells. Finding the similarity of cells using these techniques described herein can be very important in cancer research, as these techniques can determine similarities between specific cells that are rare in a general population of cells. In some cases, these similar cells may be responsible for causing metastasis. Conventional methods of analyzing genes and cells may not be capable of finding the similarity of such rare cells in a population, and thus the connections between these cells may not be uncovered. The disclosed systems and methods find connections between cells and genes that are difficult to determine due to the great number of cells and genes and the relatively small number of cells having similarities. In other words, the similarities between the cells are hidden by the number of other cells and connections. Clustering may not be suitable for determining similarities in such situations where a tiny amount of cells amongst a great number of cells have similarities.

In some embodiments, the disclosed system and computer implemented method includes leveraging the values of genes provided by single cell expression data and the structure of Random Forest decision trees to derive gene regulatory networks for individual cells. The method may include calculating split values between nodes of a decision tree, and using these split values to generate graphs indicating the relationship between genes of a single cell. Multiple graphs can be generated for individual Random Forests. Then, the graphs of the Random Forests can be consolidated into a single graph representing a gene regulatory network for an individual cell.

It is understood that the shown embodiments are extremely simplified to convey the idea of how Random Forest decision trees, bipartite graphs, and feature importance graphs are used in this disclosed system and method. However, in actual use for analyzing the relationships between cells and genes or other complex systems, thousands of Random Forest decision trees and bipartite graphs could be generated. The Random Forest decision trees and bipartite graphs could each include tens, hundreds, and thousands of nodes.

While the disclosed embodiments are discussed with the application of analyzing single cells, including RNA of cells, it is understood the disclosed embodiments can also be used with other applications. For example, the disclosed systems and methods can be used in other types of analysis involving complex relationships amongst features and/or extensive dimensionality, such as ecological, financial, actuarial, and healthcare applications. In some embodiments, the disclosed systems and methods include downstream processes, such as identification of cancer cells or relationships between genes found in cancer cells.

In one aspect, the disclosure provides a computer implemented method for identifying and quantifying relationships amongst cells and genes. The method may include receiving initial data including single cell gene expression data. The method may include generating a first random forest having a first tree and a second tree to predict a gene expression profile value of a first target gene. The method may include generating a second random forest having a third tree and a fourth tree to predict a gene expression profile value of a second target gene. The method may include generating a first bipartite graph for the first random forest from only the last two levels of the first tree and the last two levels of the second tree. The method may include generating a second bipartite graph for the second random forest from only the last two levels of the third tree and the last two levels of the fourth tree. The method may include combining the first bipartite graph with the second bipartite graph to generate a final bipartite graph representing dependencies between cells and genes. The method may include converting the final bipartite graph to embedding vectors. The method may include presenting to a user via a display of a user interface the embedding vectors.

In yet another aspect, the disclosure provides a system for identifying and quantifying relationships amongst cells and genes, comprising one or more computers and one or more storage devices storing instructions that are operable, when executed by the one or more computers, to cause the one or more computers to perform the following: (1) receive initial data including single cell gene expression data; (2) generate a first random forest having a first tree and a second tree to predict a gene expression profile value of a first target gene; (3) generate a second random forest having a third tree and a fourth tree to predict a gene expression profile value of a second target gene; (4) generate first bipartite graph for the first random forest from only the last two levels of the first tree and the last two levels of the second tree; (5) generate second bipartite graph for the second random forest from only the last two levels of the third tree and the last two levels of the fourth tree; (6) combine the first bipartite graph with the second bipartite graph to generate a final bipartite graph representing dependencies between cells and genes; (7) convert the final bipartite graph to embedding vectors; and (8) present to a user via a display of a user interface the embedding vectors.

In yet another aspect, the disclosure provides a non-transitory computer-readable medium storing software comprising instructions executable by one or more computers which, upon such execution, cause the one or more computers to identify and quantify relationships amongst cells and genes by: (1) receiving initial data including single cell gene expression data; (2) generating a first random forest having a first tree and a second tree to predict a gene expression profile value of a first target gene; (3) generating a second random forest having a third tree and a fourth tree to predict a gene expression profile value of a second target gene; (4) generating first bipartite graph for the first random forest from only the last two levels of the first tree and the last two levels of the second tree; (5) generating second bipartite graph for the second random forest from only the last two levels of the third tree and the last two levels of the fourth tree; (6) combining the first bipartite graph with the second bipartite graph to generate a final bipartite graph representing dependencies between cells and genes; (7) converting the final bipartite graph to embedding vectors; and (8) presenting to a user via a display of a user interface the embedding vectors.

The method, system, and instructions may include collecting a human tissue sample. The method may include isolating a single cell from the human tissue sample. The method may include extracting initial data from the single cell. In some embodiments, extracting initial data from a single cell includes performing single cell ribonucleic acid sequencing (scRNA-seq) on the single cell to generate first scRNA-seq data, wherein the wherein the initial data includes the first scRNA-seq data. In other words, the method may include extracting genetic material from the single cell for analysis. The data (called single cell data) produced by this process can include gene expression values of thousands of cells in the sampled tissue. In other words, the single cell data is the gene expression values representing the genetic material.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

While various embodiments are described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted.

This disclosure includes and contemplates combinations with features and elements known to the average artisan in the art. The embodiments, features, and elements that have been disclosed may also be combined with any conventional features or elements to form a distinct invention as defined by the claims. Any feature or element of any embodiment may also be combined with features or elements from other inventions to form another distinct invention as defined by the claims. Therefore, it will be understood that any of the features shown and/or discussed in the present disclosure may be implemented singularly or in any suitable combination. Accordingly, the embodiments are not to be restricted except in light of the attached claims and their equivalents. Also, various modifications and changes may be made within the scope of the attached claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a schematic diagram of a system for identifying relationships amongst cells and genes, according to an embodiment.

FIG. 2 is a schematic diagram of an overview of methods of extracting gene data.

FIG. 3 is a schematic diagram of an overview of a single cell multi-omics and further downstream processes, according to an embodiment.

FIG. 4 is a schematic diagram of generating a Random Forest for predicting each gene in a dataset, according to an embodiment.

FIG. 5 is a schematic diagram of trees from a Random Forest, according to an embodiment.

FIG. 6 is a schematic diagram of bipartite graphs generated from trees of a Random Forest, according to an embodiment.

FIG. 7 is a schematic diagram of combining bipartite graphs generated from trees of a Random Forest, according to an embodiment.

FIG. 8 is a schematic diagram of converting the combined bipartite graph to embedding vectors, according to an embodiment.

FIG. 9 shows how a Random Forest tree can be used to calculate gene significance for a cell, according to an embodiment.

FIG. 10 shows calculating the absolute value of the difference between the splitting value of a node and the gene value associated with the node, according to an embodiment.

FIG. 11 shows how bar graphs indicating gene significance for a cell for multiple Random Forest trees can be combined into a single bipartite graph for the same Random Forest, according to an embodiment.

FIG. 12 shows how gene importance graphs for multiple Random Forests can be combined to generate a gene importance graph representing a gene regulatory network (GRN) for a single cell, according to an embodiment.

FIG. 13 shows a method, according to an embodiment.

### DESCRIPTION OF EMBODIMENTS

A system and computer implemented method for identifying and quantifying relationships amongst cells and genes is disclosed. In some embodiments, the method may include collecting a human tissue sample. The method may include isolating a single cell from the human tissue sample. The method may include extracting initial data from the single cell. In some embodiments, extracting initial data from a single cell includes performing single cell ribonucleic acid sequencing (scRNA-seq) on the single cell to generate first scRNA-seq data, wherein the wherein the initial data includes the first scRNA-seq data. In other words, the method may include extracting genetic material from the single cell for analysis. The data (called single cell data) produced by this process can include gene expression values of thousands of cells in the sampled tissue. In other words, the single cell data is the gene expression values representing the genetic material.

While the disclosed embodiments are discussed with the application of analyzing single cells, including RNA of cells, it is understood the disclosed embodiments can also be used with other applications. For example, the disclosed systems and methods can be used in other types of analysis involving complex networks.

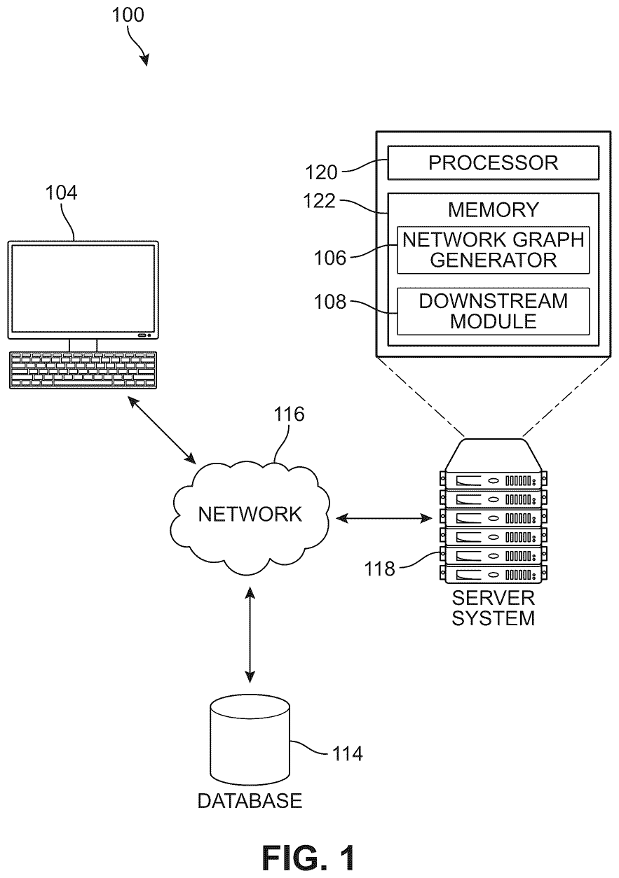

FIG. 1 is a schematic diagram of a system for identifying and quantifying relationships amongst cells and genes 100 (or system 100), according to an embodiment. System 100 may include a user and user device 102 (or device 102 or user 102). During use, a user may interact with the system to identify and quantify relationships amongst cells and genes, as well as generate graphs (e.g., bipartite graphs and gene importance graphs) visually demonstrating the relationships amongst cells and genes.

The disclosed system may include a plurality of components capable of performing the disclosed computer implemented method. For example, system 100 includes a user device 104, a computing system 118, and a database 114. Database 114 may store information about one or more cells. For example, database 114 may store databases containing information about one or more cells. In another example, database 114 may store arrays or lists of information about one or more cells.

The components of system 100 can communicate with each other through a communication network 116. For example, user device 104 may retrieve information about a cell from database 114 via communication network 116. In some embodiments, communication network 116 may be a wide area network (“WAN”), e.g., the Internet. In other embodiments, communication network 116 may be a local area network (“LAN”).

While FIG. 1 shows one user device, it is understood that one or more user devices may be used. For example, in some embodiments, the system may include two or three user devices. In some embodiments, the user devices may be computing devices used by a user. For example, user device 102 may include a smartphone or a tablet computer. In other examples, user device 102 may include a laptop computer, a desktop computer, and/or another type of computing device. The user devices may be used for inputting, processing, and displaying information. In some embodiments, a digital camera may be used to generate images used for analysis in the disclosed method. In some embodiments, the user device may include a digital camera that is separate from the computing device. In other embodiments, the user device may include a digital camera that is integral with the computing device, such as a camera on a smartphone or tablet.

As shown in FIG. 1, in some embodiments, a processor 120, a network graph generator 106 and a downstream module 108 may be hosted in a computing system 118. Generally, network graph generator 106 can generate a graph representing a gene regulatory network from input data (e.g., single cell gene expression data) to be utilized by downstream module 108 for downstream computing processes, such as processes related to identifying similarities between cells, identifying types of cells associated with types of cancer, and/or targeting certain cells for cancer treatment. Computing system 118 includes a processor 120 and a memory 122. Processor 120 may include a single device processor located on a single device, or it may include multiple device processors located on one or more physical devices. Memory 122 may include any type of storage, which may be physically located on one physical device, or on multiple physical devices. In some cases, computing system 118 may comprise one or more servers that are used to host the system.

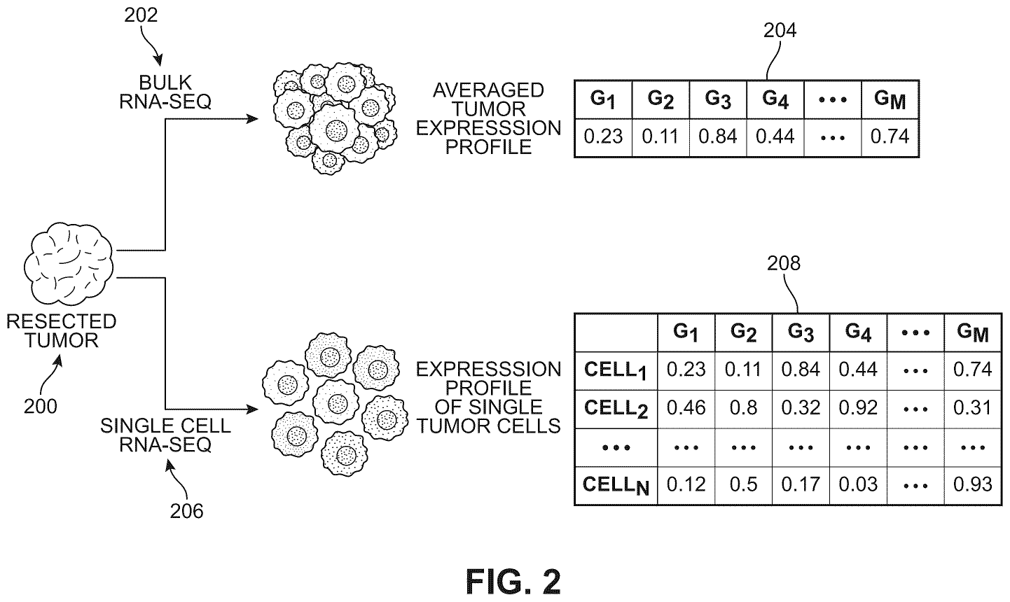

FIG. 2 is a schematic diagram of an overview of methods of extracting gene data to convey how single cell sequencing yields much more detailed information than bulk sequencing. In this example, a resected tumor sample 200 may undergo bulk RNA sequencing 202 to produce an averaged tumor expression profile. Table 204 shows an example of columns representing genes. The value for each column is an average gene expression value for all of the cells analyzed in resected tumor sample 200. Also in this example, the resected tumor sample may undergo single cell RNA sequencing 206 to produce an expression profile of single tumor cells. Table 208 shows an example of columns representing genes and rows representing individual cells from resected tumor sample 200. The value for each gene is specific to an individual cell. As discussed above, bulk sequencing produces an average genome, which is representative of broad strokes of a genome. Single cell sequencing produces genomes of individual cells that form a cell population. Table 204 next to table 208 demonstrates how much more information is extracted by single cell RNA sequencing than by bulk RNA sequencing. The advancement of single cell sequencing improves the ability to identify more granular properties of individual cells and to measure the RNA expression of a considerable amount of single cells simultaneously, resulting in greatly increasing the knowledge of cellular structure.

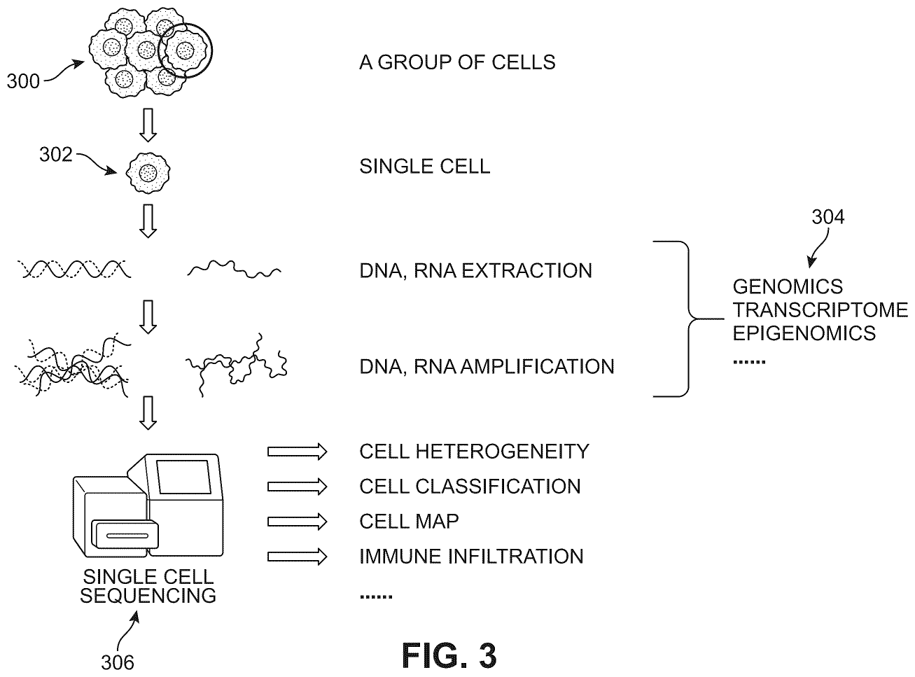

Embodiments may include single cell multi-omics and further downstream processes. FIG. 3 is a schematic diagram of an overview of a single cell multi-omics and further downstream processes, according to an embodiment. This example demonstrates how applying hyperdimensional computing and dimension reduction to extract properties from a sparse data set can be used in single cell multi-omics and further downstream processes. Single cell multi-omics can begin with single cell RNA sequencing, which can include collecting a group of cells 300, e.g., by resection. In some embodiments, the method may include collecting a human tissue sample. For example, a group of cells may be obtained from a human tissue sample. In some embodiments, the human tissue sample may be collected by resecting directly. In other embodiments, the human tissue sample may be collected by receiving an already-resected human tissue sample. Single cell RNA sequencing can further include isolating a single cell 302 from a cell population. The method may include isolating a single cell from the human tissue sample. In some embodiments, single cell RNA sequencing can include extracting, processing, and amplifying Deoxyribonucleic Acid (DNA) and RNA of each isolated cell to perform multi-omics 304, such as genomics, transcriptome, and epigenomics. The method may include performing single cell sequencing to generate data that can be used to perform downstream processes, such as determining cell heterogeneity, cell classification, generating a cell map, and identifying immune infiltration. The information generated by single cell sequencing can be sparse. The disclosed methods of hyperdimensional computing and dimension reduction can be applied to extract more granular, precise properties from the sparse data set.

In some embodiments, the method may include generating the single cell gene expression data. In some embodiments, the method may include receiving single cell gene expression data that has already been generated. The single cell gene expression data may include at least one table including rows assigned to cells and columns assigned to genes. The values in the cells describe the gene expression of each gene for the cell of the respective row.

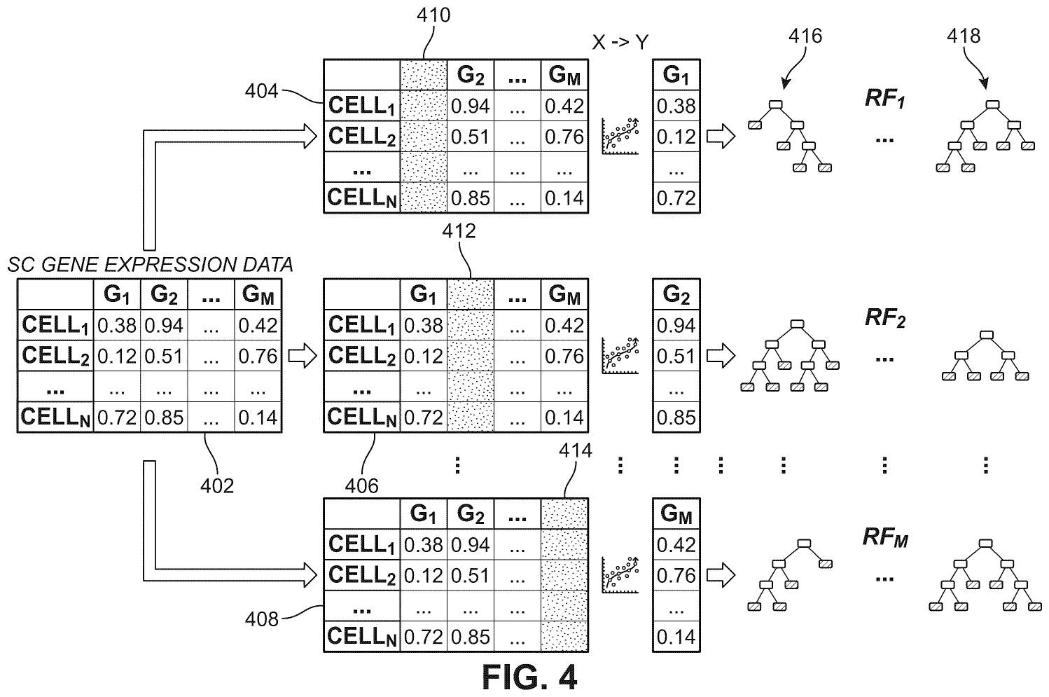

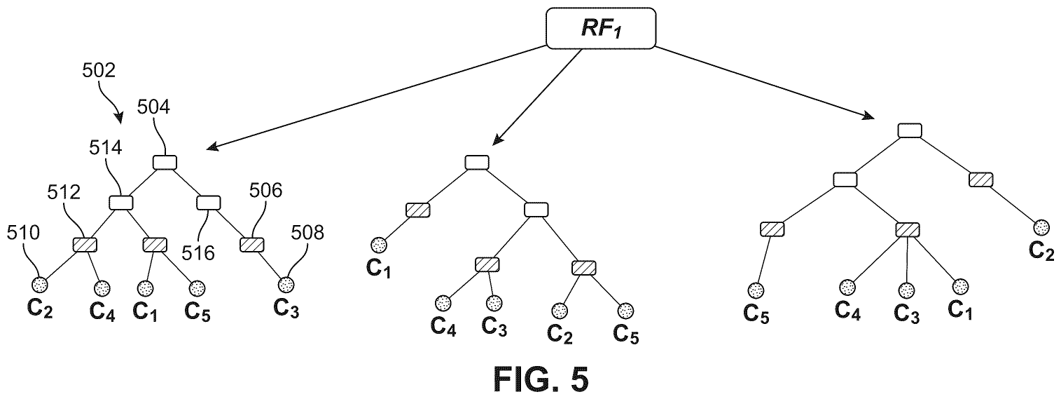

The method may include modeling the gene-gene interaction of the single cell data for each gene separately. For example, in some embodiments, a Random Forest may be generated to predict the expression profile of each target gene from profiles of all the other genes. The method may include masking a column in the table containing single cell gene expression data and then using the values in columns surrounding the masked column to generate a Random Forest to predict the values in the masked column. The purpose of generating a Random Forest is not to actually predict the values in the masked column, as these values are already available. The purpose of generating the Random Forest is to leverage the generated Random Forest trees to determine relationships amongst genes within a single cell and/or amongst genes and cells. The process of masking columns and generating a Random Forest for predicting the values in the masked column may be performed for one or more genes in the table containing single cell expression data. FIG. 4 is a schematic diagram of generating a Random Forest for predicting each gene in a dataset, according to an embodiment. Random Forest unveils the significance of various features in predicting values. For instance, in the realm of forecasting a gene's value, Random Forest discerns the pivotal role of other genes in this predictive process. FIG. 4 shows a table 402 containing single cell expression data. The column 410 for gene G₁ is masked in table 404 (even though the values for gene G₁ are actually known) and Random Forest RF₁ is generated for predicting the values in masked column 410. The column 412 for gene G₂ is masked (even though the values for gene G₂ are actually known) in table 406 and Random Forest RF₂ is generated for predicting the values in masked column 412. The column 414 for gene G_{M} is masked (even though the values for gene G_{M} are actually known) in table 408 and Random Forest RF₃ is generated for predicting the values in masked column 414. In FIG. 4, the images of Random Forest trees (e.g., tree 416 and tree 418) representing Random Forest RF₁, Random Forest RF₂, and Random Forest RF₃ are merely representative and do not show full details of the Random Forests generated. FIG. 5, discussed below, provides more detail of exemplary Random Forest trees in Random Forest RF₁. It is understood that Random Forest RF₁, Random Forest RF₂, and Random Forest RF₃ can each include more trees and the trees can include the type of details (i.e., the cell nodes discussed below) shown in FIG. 5.

FIG. 5 is a schematic diagram of Random Forest trees representing Random Forest RF₁, according to an embodiment. A Random Forest tree 502 includes a root node 504 and other nodes connected to the root node by branches (i.e., straight lines). Nodes other than the root node can be connected to other nodes by branches as well. At each node connected directly to the root node (e.g., nodes 514 and 516), another feature (e.g., a gene in this embodiment) will be selected to be the next node connected by a branch to the node closest to the root node. For example, nodes 514 and 516 may be selected based on feature importance. The selection may be made during the training phase of Random Forest. The selection may be made based on feature importance. Following down the line of branches from the root node, each subsequent node is selected in this fashion until there is a node that does not split. Random Forest trees include leaf nodes (e.g., leaf node 506 and leaf node 512), which are nodes that do not split. The Random Forest trees may also include a last level of nodes (shown as circular nodes) representing individual cells (e.g., C₁, C₂, C₃, and C₄). For example, reference number 508 indicates a node representing C₃ and reference number 510 indicates a node representing C₂. In Random Forest tree 502, node 508 is connected to leaf node 506. Each leaf node includes cells showing more similar gene dependency in predicting the target gene.

In some embodiments, the method may include operations related to determining similarities between cells. For example, the method may include modeling the last layer of each Forest θ (including leaves and connected cells) with an undirected bipartite graph LG_{θ}=(V_{θ}, E_{θ}). In other words, the method may include generating bipartite graphs from only the last two levels of nodes of the Random Forest trees. For example, in FIG. 5, Random Forest tree 502 may include the following last two levels: a last level including the circular nodes representing cells (including node 508) and a second to last level including rectangular striped nodes (including node 506 and other nodes directly connected to the circular nodes). After training a random forest, samples (in this situation, cells) are landed in various leaf nodes. Hence, two cells Cᵢ and Cⱼ are placed in common or closer leaf nodes throughout this model if their gene-gene interactions are generally quite comparable. Hence, in this phase, a relation graph is extracted by connecting cells to their relevant leaf nodes across all forests and then eliminating the rest of the trees. By doing so, the resulting relation graph can model how each regulatory prediction model (i.e., each random forest regression model) relates to the others as well as how the decision trees inside each random forest interact with one another.

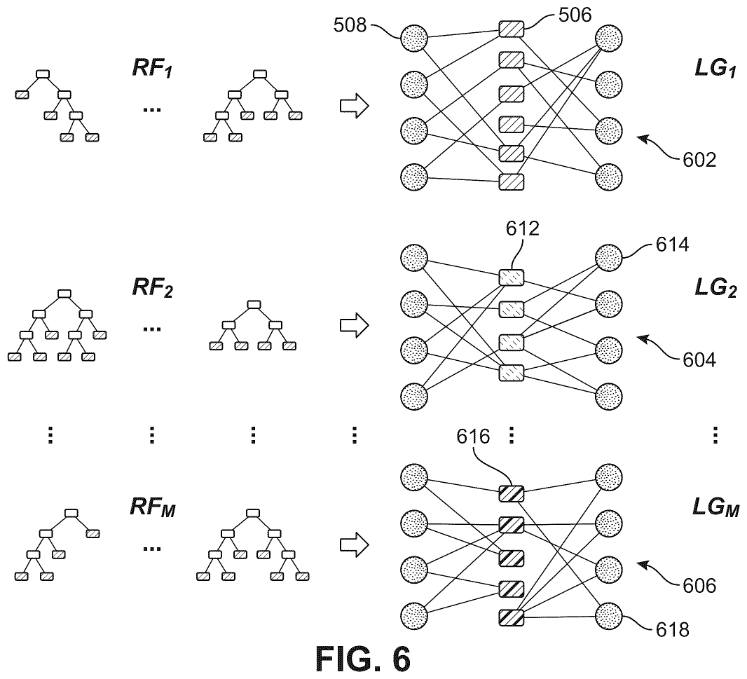

FIG. 6 shows exemplary bipartite graphs 602, 604, and 606 generated from Random Forest RF₁, Random Forest RF₂, and Random Forest RF₃, respectively. Similar to FIG. 4, the images of Random Forest trees representing Random Forest RF₁, Random Forest RF₂, and Random Forest RF₃ in FIG. 6 are merely representative and do not show full details of the Random Forests generated. It is understood that the images of Random Forest trees representing Random Forest RF₁, Random Forest RF₂, and Random Forest RF₃ in FIG. 6 can include more nodes, including more leaf nodes and/or cell nodes. FIG. 6 does not show Random Forest tree 502 of Random Forest RF₁, but it is understood that Random Forest RF₁ includes Random Forest tree 502. Thus, the last two levels of nodes of the Random Forest tree 502 are included in bipartite graph 602. In bipartite graphs 602, 604, and 606, each leaf node (e.g., rectangular nodes) is a super node connecting the most similar cells (e.g., circular nodes) to each other. For example, in bipartite graph 602, the leaf nodes may include leaf node 506 from the second to last level of Random Forest tree 502 and the cells may include node 508 from the last level of Random Forest tree. In bipartite graph 604, leaf node 612 is an example of a super node connecting cells, such as cell node 614, to other super nodes. In bipartite graph 606, leaf node 616 is an example of a super node connecting cells, such as cell node 618, to other super nodes.

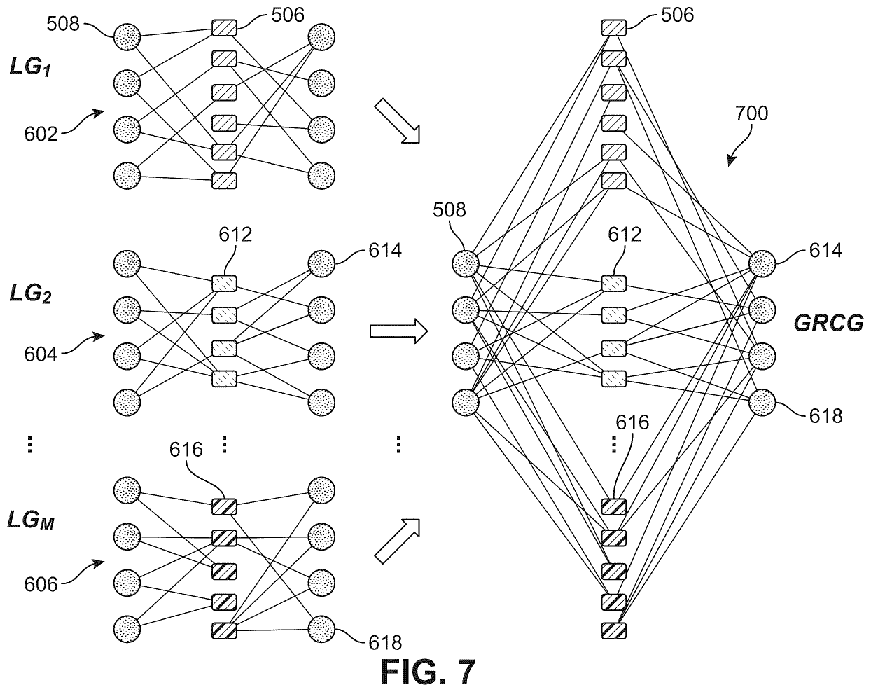

The method may include merging all the generated bipartite graphs (e.g., bipartite graphs 602, 604, and 606) using their shared cells landed in leaves. Since identical cells are employed for each tree, these cell nodes are shared among various leaf nodes across different trees. Therefore, these shared points (e.g., cells) serve as the linkage to connect various extracted graphs from diverse trees. The merged graph can be called a Gene Regulatory Cell Graph (GRCG). FIG. 7 shows an example of a GRCG 700. As in FIGS. 5 and 6, the circular nodes in FIG. 7 represent individual cells (e.g., C₁, C₂, C₃, and C₄). GRCG 700 shows the connections (e.g., represented by straight lines) between rectangular nodes (representing genes) and circular nodes (representing cells). These connections show dependencies between cells and genes.

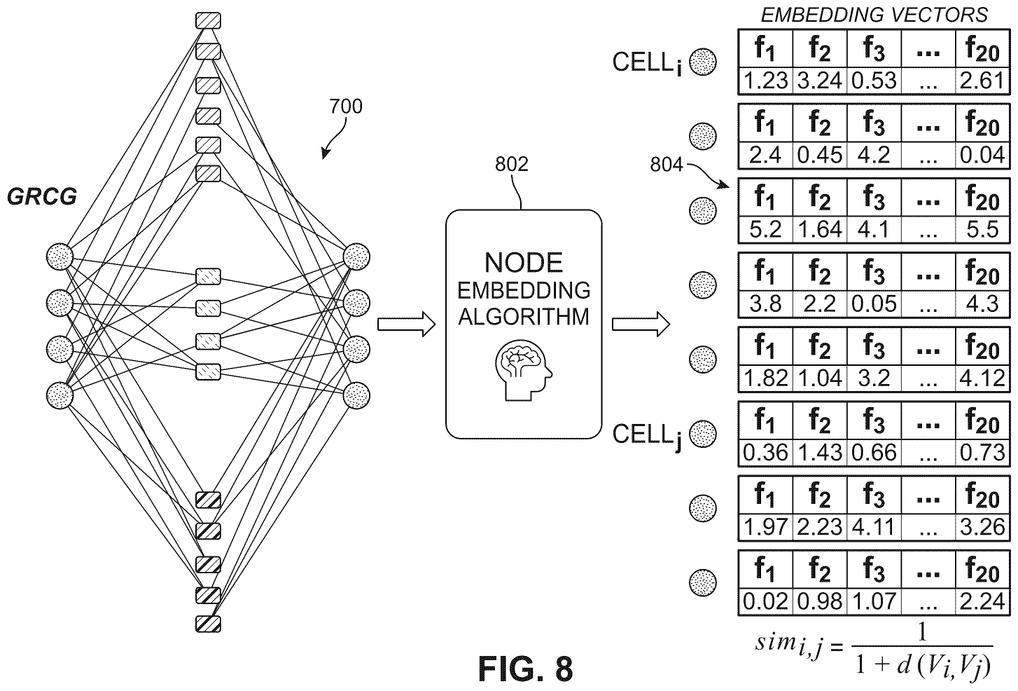

As shown in FIG. 8, the method may include applying a node embedding algorithm (e.g., Node2Vec, Fast Random Projection, NodePiece, LINE algorithm, etc.) 802 to GRCG 700 to generate an embedding representation of the cell nodes in GRCG 700. In general, node embedding algorithms can compute a vector representation of each node and each vertex or edge based on random walks in the graph. Output 804 of the embedding of GRCG 700 may include columns representing new feature vectors for each cells (e.g., cells represented by cell nodes 508, 614, and 618 in FIGS. 5 and 6). Output 804 can provide a biologically meaningful embedding of cells. For example, output 804 may include: (a) gene ranking (hierarchy in tree branching) and (b) global interaction among all the genes (branches in tree structures). Output 804 may include columns representing new feature vectors for each cells. The method can include computing the similarity between any pair of cells like Cellᵢ and Cellⱼ as standard Euclidean distance between their learned embedding features of these two cells. For example, the method may include applying the following equation to compute the similarity between Cellᵢ and Cellⱼ:

$${sim}_{i , j} = \frac{1}{1 + d ( {V}_{i} , {V}_{j} )} ,$$

where
d(Vᵢ, Vⱼ): Euclidean distance between embedding vectors Vᵢ and Vⱼ

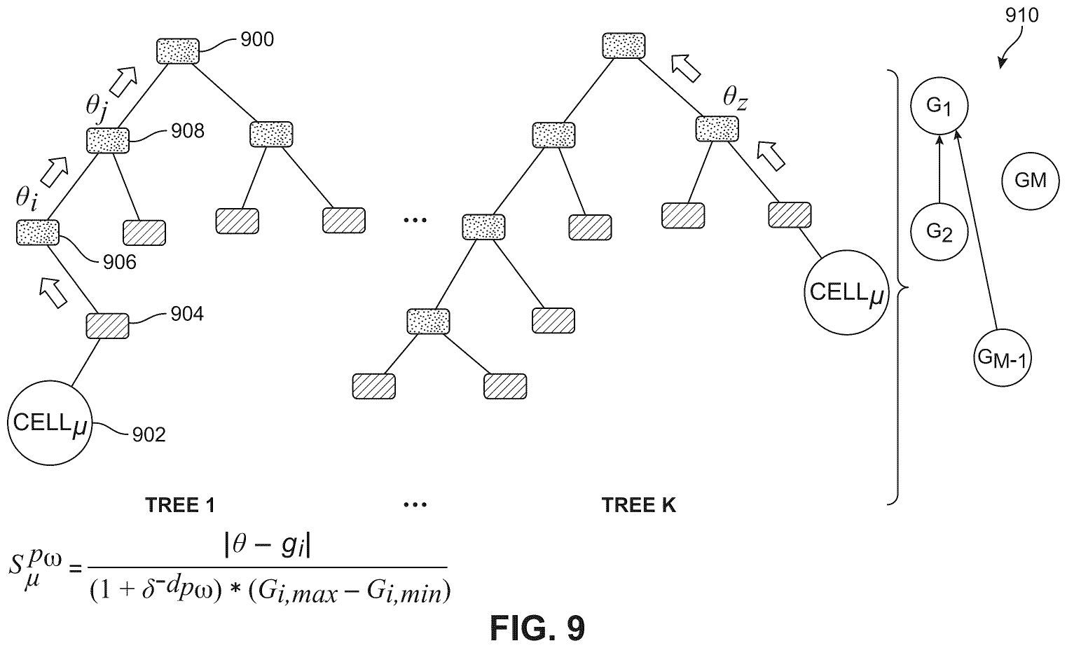

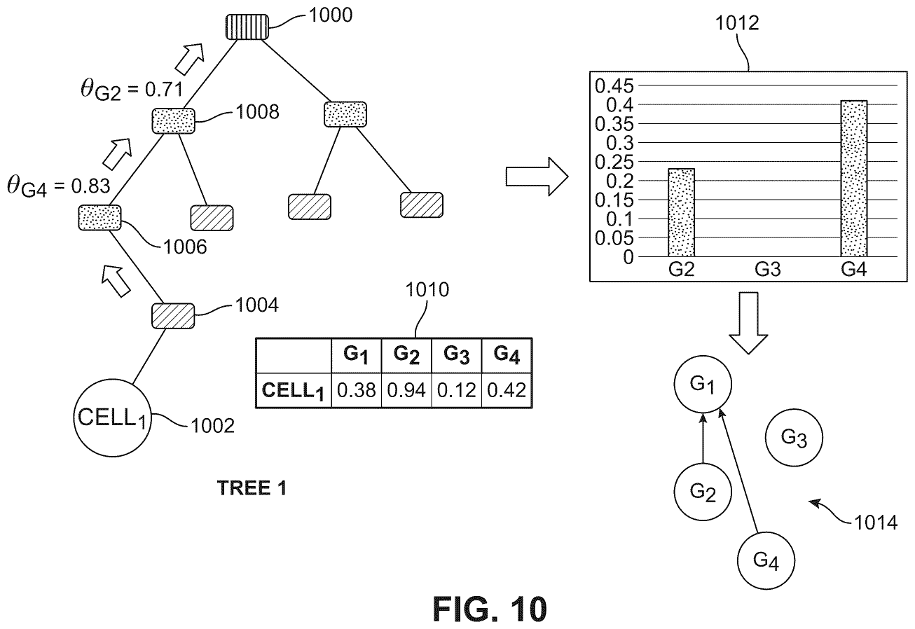

In some embodiments, the method may include operations related to generating a gene regulatory network for an individual cell using scRNA-seq data. For example, the method may include, after generating a Random Forest, as discussed above with respect to FIGS. 4-5, modeling the gene-gene interaction of entire single cell data for each gene separately. In some embodiments, as shown in FIGS. 9-10, the generated Random Forest trees may provide a structure for guiding the calculation of values indicating the significance of various genes within a gene regulatory network for a cell. In FIGS. 9-10, the rectangular nodes represent genes and the circular nodes represent cells. The Random Forest shown in FIG. 9 may include k trees, where each tree tᵢ has depth dᵢ. The method may include moving upward from node 902 representing cell μ to reach root node 900. This part of the method, as well as the embodiment discussed with respect to FIG. 10, focuses on the entire tree with more emphasis (e.g., weight) on the nodes closest to the root node. As discussed in more detail below, the absolute difference between the values of the nodes in the Random Forest trees and the values of the genes per cell in each input sequencing value can be considered as part of determining the importance each gene has for each individual cell.

FIG. 9 shows Tree 1 with leaf nodes (rectangular striped nodes), including leaf node 904. The leaf nodes are connected directly to the last layer (which includes node 902), and act as a bucket. Thus, there is no split value between nodes 904 and 906. FIG. 9 shows intermediary nodes 906 and 908 between root node 900 and leaf node 904. Each intermediary node may have a split value θ. For example, intermediary node 906 may have a split value θi and intermediary node 908 may have a split value θj. For each node, p_{ω}, there exists a split value θ. The significance of gene I at node p_{ω} can be expressed as follows:

$${S}_{μ}^{{p}_{ω}} = \frac{\vert θ - {g}_{i} \vert}{( 1 + {δ}^{- {d}_{{p}_{ω}}} ) * ( {G}_{i , max} - {G}_{i , min} )} ,$$

where Pᵢ^{μ}={p₁, p₂, . . . , p_{γ}} is the sequence of nodes from cell μ to the root of a tree and the expression value for gene Gᵢ is gᵢ. In this equation, dₚ _{_{ω}} is the depth of node p_{ω}, gᵢ is the expression value of gene Gᵢ, δ is a tuning parameter, G_{i,max} and G_{i,min} denote the maximum and minimum values of gene Gᵢ across all cells, respectively. The closer a node is to the root node, the more significant the node is. Thus, this position of nodes (with respect to the position of the root node) is factored into the above equation by dₚ_{_{ω}}. As discussed in more detail with respect to FIG. 10, the split values may be used to make a graph 910 representing a gene regulatory network. In the case of FIG. 9, the gene regulatory network is based on Random Forest Tree 1 through Tree K.

FIG. 10 shows an embodiment in which the method includes calculating the absolute value of the difference between the splitting value of a node and the gene value associated with the node. FIG. 10 shows Tree 1 with leaf nodes (rectangular nodes), including leaf node 1004. The leaf nodes are connected directly to the last layer (including node 1002 representing Cell₁), and act as a bucket or container. FIG. 10 shows intermediary nodes 1006 and 1008 between root node 1000 and leaf node 1004. Each intermediary node may have a split value θ that may be provided by the process of generating a Random Forest. For example, intermediary node 1006 may have a split value θ_{G4} and intermediary node 1008 may have a split value θ_{G2}. θ_{G4} is shown as having a value of 0.83 and θ_{G2} is shown as having a value of 0.71. Table 1010 is a row taken from a bigger table that is similar to Table 402. For simplicity in explanation only a single row with 4 genes and 1 cell is shown in Table 1010. These are values that can be produced by single cell sequencing of a tissue sample and provided as input to form the Random Forest decision trees. It is understood that in actual use the table and the Random Forest decision trees may include thousands of genes.

As previously mentioned, the method may include calculating the absolute value of the difference between the splitting value of a node and the gene value associated with the node. For example, starting the lowest intermediary node on the branch between root node 1000 and node 1002, the absolute value of the difference between the split value of intermediary node 1006 (θ_{G4}=0.83) and the value assigned to G₄ (0.42) in Table 1010 is shown in bar graph 1012 as 0.41. Moving to intermediary node 1008, the absolute value of the difference between the split value of intermediary node 1008 (θ_{G2}=0.71) and the value assigned to G₂ (0.94) in Table 1010 is shown in bar graph 1012 as 0.23. The values in bar graph 1012 indicate the significance of the genes in the tree for Cell₁. As visually represented by bar graph 1012 the value for G₄ is bigger than the value for G₂. Accordingly, G₄ is more significant, or has more influence, in Cell₁ than G₂ does. The value for G₃ in bar graph 1012 is zero because G₃ did not participate in this pass of Tree 1. In some embodiments, a threshold value may determine whether or not a gene participates in a pass. For example, in some embodiments, a gene may be considered as participating if its value is in the top 20% of values.

FIG. 10 shows a gene importance graph 1014. The method may include computing the total significance of gene Gᵢ in prediction of cell μ as the sum of all S_{μ}^{p}^{_{ω}} for all p_{ω} across all T trees, as follows:

$${S}_{μ}^{{G}_{i}} = {\sum }_{j = 1}^{k} {\sum }_{{p}_{ω}}^{{P}_{i}^{μ}} {S}_{μ}^{{p}_{ω}}$$

This sum can be used to generate a sub-graph, such as a feature importance graph, (e.g., gene importance graph 1014) indicating how other genes affect gene Gᵢ. Gene importance graph 1014 shows how genes G₂, G₃, and G₄ affect G₁. In gene importance graph 1014, G₃ is shown as having no affect on G₁ because G₃ is not in the top 20% of values. As previously mentioned, for simplicity the example in FIG. 10 only shows 4 genes. However, in actual use, a Random Forest may involve thousands of genes that are also included in gene importance graphs created based on the respective Random Forest.

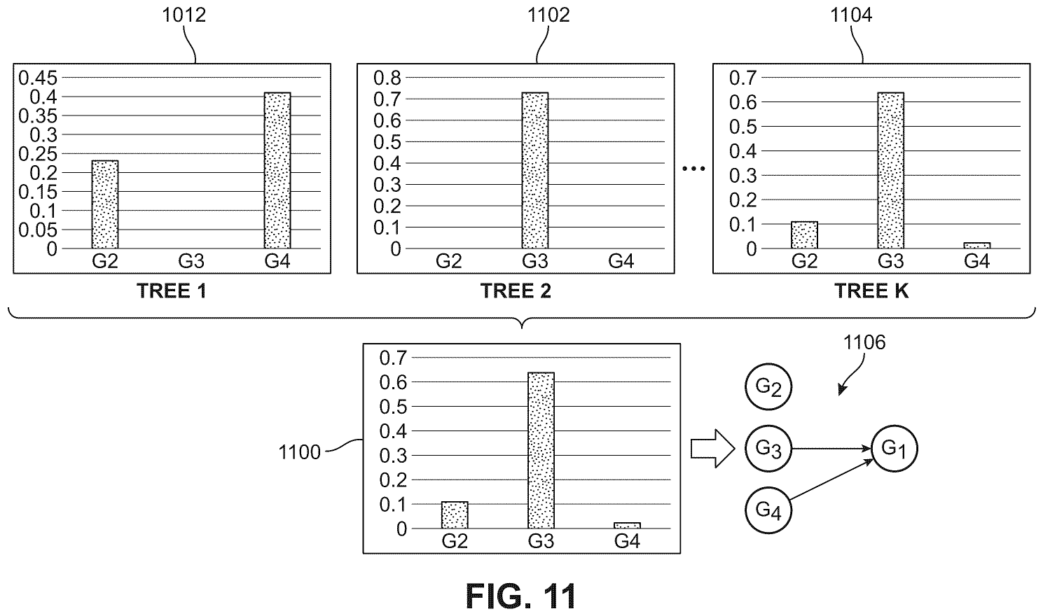

FIG. 11 shows how bar graphs indicating gene significance for a cell for multiple Random Forest decision trees can be combined into a single graph for the same Random Forest, according to an embodiment. FIG. 11 shows bar graph 1012 for Tree 1, as well as table 1102 for Tree 2 and table 1104 for Tree k. Bar graph 1012, table 1102, and table 1104 may all be generated for the same cell from the same Random Forest. Bar graph 1012, table 1102, and table 1104 may be combined into table 1100. Then, a graph 11060 can be created based on table 1100 in the manner discussed with respect to FIG. 10.

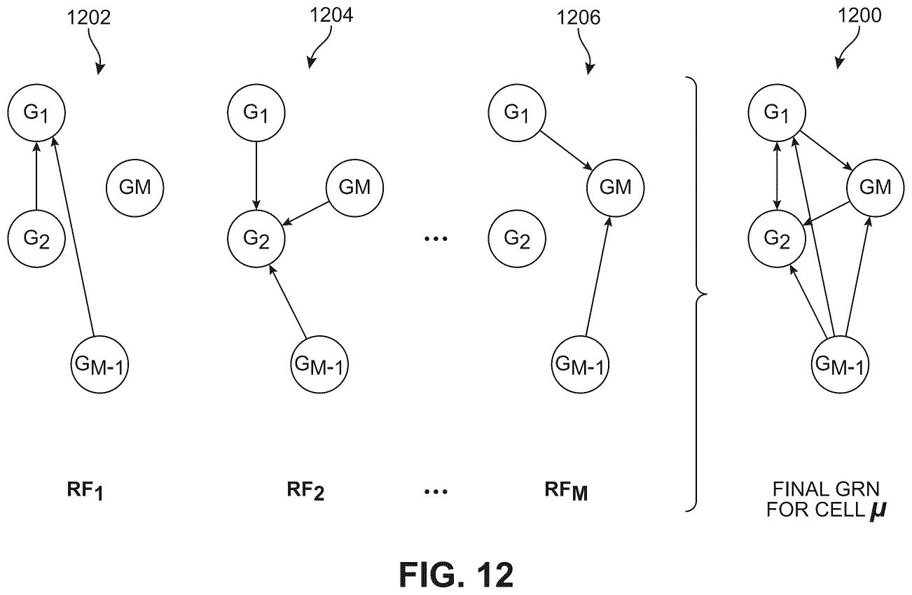

FIG. 12 shows how gene importance graphs for multiple Random Forests can be combined to generate a single gene importance graph representing a gene regulatory network (GRN) for a single cell, according to an embodiment. Graph 1202 represents Random Forest 1. In graph 1202, G₁ is a dependent variable (target output of Random Forest 1 regression model) and the rest of the genes (i.e., G₂, . . . , G_{M-1}, G_{M}) are independent variables. Graph 1204 represents Random Forest 2. In graph 1204, G₂ is a dependent variable (target output of Random Forest 2 regression model) and the rest of the genes (i.e., G₁, . . . , G_{M-1}, G_{M}) are independent variables. Graph 1206 represents Random Forest M. In graph 1206, G_{M} is a dependent variable (target output of Random Forest M regression model) and the rest of the genes (i.e., G₁, G₂, . . . , G_{M-1}) are independent variables. Graph 1200 is a final gene importance graph representing Random Forests 1 through M for cell μ. In Graph 1200, the connections (i.e., arrows) between genes in graph 1202, graph 1204, and graph 1206 are consolidated. This final gene importance graph represents the influence of each gene per cell. In other words, the final gene importance graph represents a gene regulatory network for an individual cell.

The final gene importance graphs can be extremely useful in downstream processes. In some embodiments, the final gene importance graphs for various cells can be compared. For example, a graph for a normal cell can be compared with a graph for a cancer cell. This comparison can help determine which new links between genes are generated in cancer cells. This information can be used to develop drugs to switch off the genes that influence cancer. In comparison to conventional cancer treatments that destroy healthy cells as collateral damage to trying to destroy healthy cells, this type of cancer treatment would preserve healthy cells.

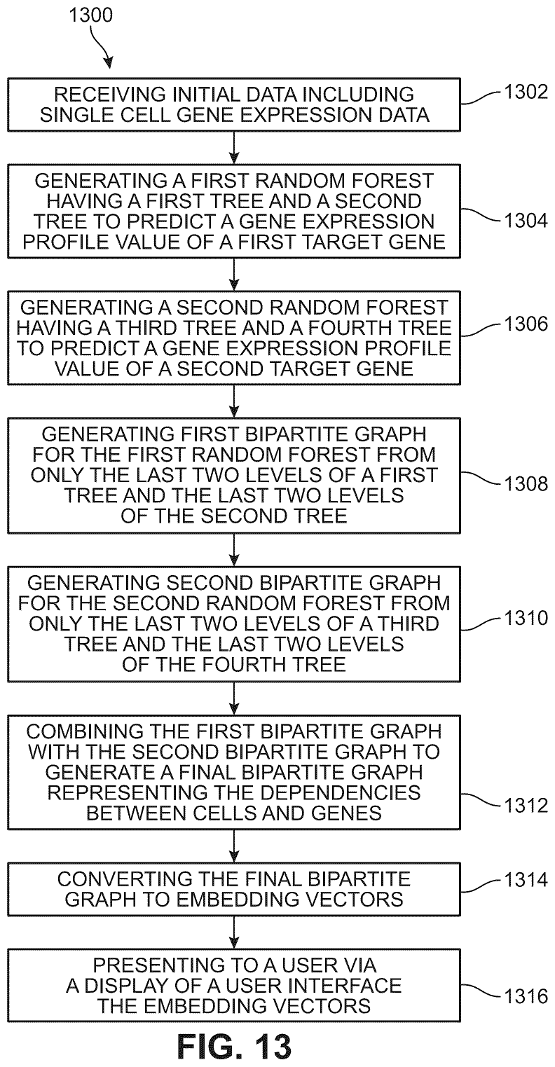

FIG. 13 shows a method 1300, according to an embodiment. In some embodiments, the computer implemented method for identifying and quantifying relationships amongst cells and genes may include receiving initial data including single cell gene expression data (operation 1302). The method may include generating a first random forest having a first tree and a second tree to predict a gene expression profile value of a first target gene (operation 1304). The method may include generating a second random forest having a third tree and a fourth tree to predict a gene expression profile value of a second target gene (operation 1306). The method may include generating a first bipartite graph for the first random forest from only the last two levels of the first tree and the last two levels of the second tree (operation 1308). The method may include generating a second bipartite graph for the second random forest from only the last two levels of the third tree and the last two levels of the fourth tree (operation 1310). The method may include combining the first bipartite graph with the second bipartite graph to generate a final bipartite graph representing dependencies between cells and genes (operation 1312). The method may include converting the final bipartite graph to embedding vectors (operation 1314). The method may include presenting to a user via a display of a user interface the embedding vectors (operation 1316).

The method may include determining a Euclidean distance between embedding vectors converted from a final bipartite graph to embedding vectors associated with a second cell to determine the similarity between the first cell and the second cell. For example, determining this distance may be performed according to the embodiment discussed with respect to FIG. 8 above. Generating a first random forest having a first tree and a second tree to predict a gene expression profile value of a first target gene may include masking known values for the first target gene in the single cell expression data, for example, as discussed above with respect to FIG. 4 above. In some embodiments, the first tree and the second tree may each have a root node, a bottom node representing a cell, and intermediary nodes between the root node and the bottom node. The method may include calculating split values between the intermediary nodes of the first tree, for example, as described with respect to FIGS. 9-10 above. The method may include using the calculated split values of the first tree to generate a first gene importance graph indicating the relationship between genes of a single cell based on the first tree, for example, as described with respect to FIGS. 9-11 above. The method may include calculating split values between the intermediary nodes of the second tree; using the calculated split values of the second tree to generate graphs indicating the relationship between genes of the single cell based on the second tree; and using the calculated split values of the first tree to generate a second gene importance graph indicating the relationship between genes of a single cell based on the first tree, for example, as described with respect to FIGS. 9-11 above. The method may include consolidating the first gene importance graph and the second gene importance graph to create a final gene importance graph, for example, as described with respect to FIG. 12 above.

Embodiments may include a non-transitory computer-readable medium (CRM) storing software comprising instructions executable by one or more computers which, upon such execution, cause the one or more computers to perform the disclosed methods. Non-transitory CRM may refer to a CRM that stores data for short periods or in the presence of power such as a memory device or Random Access Memory (RAM). For example, a non-transitory computer-readable medium may include storage components, such as, a hard disk (e.g., a magnetic disk, an optical disk, a magneto-optic disk, and/or a solid state disk), a compact disc (CD), a digital versatile disc (DVD), a floppy disk, a cartridge, and/or a magnetic tape.

Embodiments may also include one or more computers and one or more storage devices storing instructions that are operable, when executed by the one or more computers, to cause the one or more computers to perform the disclosed methods.

Cloud computing environment can include, for example, an environment that hosts the policy management service. The cloud computing environment may provide computation, software, data access, storage, etc. services that do not require end-user knowledge of a physical location and configuration of system(s) and/or device(s) that hosts the policy management service. For example, a cloud computing environment may include a group of computing resources (referred to collectively as “computing resources” and individually as “computing resource”).

While various embodiments are described, the description is intended to be exemplary, rather than limiting and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

This disclosure includes and contemplates combinations with features and elements known to the average artisan in the art. The embodiments, features, and elements that have been disclosed may also be combined with any conventional features or elements to form a distinct invention as defined by the claims. Any feature or element of any embodiment may also be combined with features or elements from other inventions to form another distinct invention as defined by the claims. Therefore, it will be understood that any of the features shown and/or discussed in the present disclosure may be implemented singularly or in any suitable combination. Accordingly, the embodiments are not to be restricted except in light of the attached claims and their equivalents. Also, various modifications and changes may be made within the scope of the attached claims.

We claim:

1. A computer implemented method for identifying and quantifying relationships amongst cells and genes, comprising:
receiving initial data including single cell gene expression data;
generating a first random forest having a first tree and a second tree to predict a gene expression profile value of a first target gene;
generating a second random forest having a third tree and a fourth tree to predict a gene expression profile value of a second target gene;
generating first bipartite graph for the first random forest from only the last two levels of the first tree and the last two levels of the second tree;
generating second bipartite graph for the second random forest from only the last two levels of the third tree and the last two levels of the fourth tree;
combining the first bipartite graph with the second bipartite graph to generate a final bipartite graph representing dependencies between cells and genes; and
converting the final bipartite graph to embedding vectors representing features of a first cell.

2. The method of claim 1, further including determining a Euclidean distance between the embedding vectors converted from the final bipartite graph to embedding vectors associated with a second cell to determine the similarity between the first cell and the second cell.

3. The method of claim 1, wherein generating a first random forest having a first tree and a second tree to predict a gene expression profile value of a first target gene includes masking known values for the first target gene in the single cell expression data.

4. The method of claim 3, wherein the first tree and the second tree each have a root node, a bottom node representing a cell, and intermediary nodes between the root node and the bottom node, and further comprising:
calculating split values between the intermediary nodes of the first tree.

5. The method of claim 4, further comprising:
using the calculated split values of the first tree to generate a first gene importance graph indicating the relationship between genes of a single cell based on the first tree.

6. The method of claim 5, further comprising:
calculating split values between the intermediary nodes of the second tree;
using the calculated split values of the second tree to generate graphs indicating the relationship between genes of the single cell based on the second tree; and
using the calculated split values of the first tree to generate a second gene importance graph indicating the relationship between genes of a single cell based on the first tree.

7. The method of claim 6, further comprising:
consolidating the first gene importance graph and the second gene importance graph to create a final gene importance graph.

8. A system for identifying and quantifying relationships amongst cells and genes, comprising one or more computers and one or more storage devices storing instructions that are operable, when executed by the one or more computers, to cause the one or more computers to perform the following:
receive initial data including single cell gene expression data;
generate a first random forest having a first tree and a second tree to predict a gene expression profile value of a first target gene;
generate a second random forest having a third tree and a fourth tree to predict a gene expression profile value of a second target gene;
generate first bipartite graph for the first random forest from only the last two levels of the first tree and the last two levels of the second tree;
generate second bipartite graph for the second random forest from only the last two levels of the third tree and the last two levels of the fourth tree;
combine the first bipartite graph with the second bipartite graph to generate a final bipartite graph representing dependencies between cells and genes; and
convert the final bipartite graph to embedding vectors representing features of a first cell.

9. The system of claim 8, wherein the instructions are further operable, when executed by the one or more computers, to cause the one or more computers to perform the following:
determine a Euclidean distance between the embedding vectors converted from the final bipartite graph to embedding vectors associated with a second cell to determine the similarity between the first cell and the second cell.

10. The system of claim 8, wherein generating a first random forest having a first tree and a second tree to predict a gene expression profile value of a first target gene includes masking known values for the first target gene in the single cell expression data.

11. The system of claim 10, wherein the first tree and the second tree each have a root node, a bottom node representing a cell, and intermediary nodes between the root node and the bottom node, and wherein the instructions are further operable, when executed by the one or more computers, to cause the one or more computers to perform the following:
calculate split values between the intermediary nodes of the first tree.

12. The system of claim 11, wherein the instructions are further operable, when executed by the one or more computers, to cause the one or more computers to perform the following:
use the calculated split values of the first tree to generate a first gene importance graph indicating the relationship between genes of a single cell based on the first tree.

13. The system of claim 12, wherein the instructions are further operable, when executed by the one or more computers, to cause the one or more computers to perform the following:
calculate split values between the intermediary nodes of the second tree;
use the calculated split values of the second tree to generate graphs indicating the relationship between genes of the single cell based on the second tree; and
use the calculated split values of the first tree to generate a second gene importance graph indicating the relationship between genes of a single cell based on the first tree.

14. The system of claim 13, wherein the instructions are further operable, when executed by the one or more computers, to cause the one or more computers to perform the following:
consolidate the first gene importance graph and the second gene importance graph to create a final gene importance graph.

15. A non-transitory computer-readable medium storing software comprising instructions executable by one or more computers which, upon such execution, cause the one or more computers to identify and quantify relationships amongst cells and genes by:
receiving initial data including single cell gene expression data;
generating a first random forest having a first tree and a second tree to predict a gene expression profile value of a first target gene;
generating a second random forest having a third tree and a fourth tree to predict a gene expression profile value of a second target gene;
generating first bipartite graph for the first random forest from only the last two levels of the first tree and the last two levels of the second tree;
generating second bipartite graph for the second random forest from only the last two levels of the third tree and the last two levels of the fourth tree;
combining the first bipartite graph with the second bipartite graph to generate a final bipartite graph representing dependencies between cells and genes; and
converting the final bipartite graph to embedding vectors representing features of a first cell.

16. The non-transitory computer-readable medium storing software of claim 15, wherein the instructions are further operable, when executed by the one or more computers, to cause the one or more computers to perform the following:
determining a Euclidean distance between the embedding vectors converted from the final bipartite graph to embedding vectors associated with a second cell to determine the similarity between the first cell and the second cell.

17. The non-transitory computer-readable medium storing software of claim 15, wherein generating a first random forest having first tree and a second tree to predict a gene expression profile value of a first target gene includes masking known values for the first target gene in the single cell expression data.

18. The non-transitory computer-readable medium storing software of claim 15, wherein the instructions are further operable, when executed by the one or more computers, to cause the one or more computers to perform the following:
calculating split values between the intermediary nodes of the first tree.

19. The non-transitory computer-readable medium storing software of claim 18, wherein the instructions are further operable, when executed by the one or more computers, to cause the one or more computers to perform the following:
use the calculated split values of the first tree to generate a first gene importance graph indicating the relationship between genes of a single cell based on the first tree.

20. The non-transitory computer-readable medium storing software of claim 19, wherein the instructions are further operable, when executed by the one or more computers, to cause the one or more computers to perform the following:
calculating split values between the intermediary nodes of the second tree;
using the calculated split values of the second tree to generate graphs indicating the relationship between genes of the single cell based on the second tree;
using the calculated split values of the first tree to generate a second gene importance graph indicating the relationship between genes of a single cell based on the first tree; and
consolidating the first gene importance graph and the second gene importance graph to create a final gene importance graph.
